(19) United States
(12) Patent Application Publication
(10) Pub. No.: US 2023/0019451 A1
(43) Pub. Date: Jan. 19, 2023

(54) DRESSING BAND

(71) Applicant: OPILEX CO., LTD., Busan (KR)

(72) Inventor: Ji Hyeok YOON, Busan (KR)

(73) Assignee: OPILEX CO., LTD., Busan (KR)

(21) Appl. No.: 17/864,377

(22) Filed: Jul. 13, 2022

(30) Foreign Application Priority Data
Jul. 13, 2021 (KR) 10-2021-0091755
Aug. 5, 2021 (KR) 10-2021-0103351

(51) Int. Cl. A61F 13/02

(52) U.S. Cl. CPC A61F 13/0266; A61F 13/0206; A61F 13/024

(57) ABSTRACT

The present invention relates to a dressing band including a sheet portion whose lower surface comes in close contact with a user's body, an adhesive portion which is applied on the lower surface of the sheet portion and attached to the body, a gauze portion which is attached to the lower surface of the sheet portion and comes in close contact with an affected area of the body, a protective film portion which is attached to a lower surface of the adhesive portion to cover the gauze portion and is separated from the adhesive portion during attachment of the sheet portion to the body, and a grip portion which protrudes from one side of the sheet portion in order to be gripped by the user and is rotatably connected to the one side of the sheet portion with respect to a fold line.

### CROSS-REFERENCE TO RELATED APPLICATION

This application claims priority to and the benefit of Korean Patent Application No. 10-2021-0091755, filed on Jul. 13, 2021 and Korean Patent Application No. 10-2021-0103351, filed on Aug. 5, 2021 the disclosures of which are incorporated herein by reference in their entireties.

### BACKGROUND

### 1. Field of the Invention

The present invention relates to a dressing band, and more particularly, to a dressing band that allows a sheet portion to be easily peeled off, secures hygiene and safety, and allows various pieces of information to be easily checked.

### 2. Discussion of Related Art

Generally, dressing bands are used to protect a wound site or prevent infection thereof in homes, hospitals, or the like, and various forms of dressing bands are used according to the purposes of use.

Such a dressing band is made of a sheet which has an adhesive applied on one surface to allow for attachment to the user's body, gauze which is attached to one surface of the sheet to come in close contact with the user's skin, and a protective film which is attached to the adhesive of the sheet to cover the gauze, and the surface of the sheet on which the adhesive is applied is attached to the user's skin in a state in which the protective film has been peeled off

However, regarding the conventional dressing bands, there are concerns that contaminants (bacteria, foreign matter, etc.) on the adhesive may be transferred to a wound and cause infection of the wound when the dressing band is attached to the user's skin, it is unhygienic because the adhesive often gets on a hand during the process of peeling off the dressing band, and there is a risk of forming an additional wound on the skin or causing infection of the wound in the process of peeling off the dressing band from the skin. Also, the dressing band is left on for a long period of time in some cases due to a difficulty of accurately checking the time of which the dressing band has been attached, and there is concern about causing additional wounds (skin problems or the like).

Korean Utility Model Publication No. 20-2013-0002638 (published on May 3, 2013), which is the related art of the present invention, discloses a dressing band.

### SUMMARY OF THE INVENTION

The present invention is directed to providing a dressing band that allows a sheet portion to be easily peeled off, secures hygiene and safety, and allows various pieces of information to be easily checked.

The present invention provides a dressing band including a sheet portion whose lower surface comes in close contact with a user's body, an adhesive portion which is applied on the lower surface of the sheet portion and attached to the body, a gauze portion which is attached to the lower surface of the sheet portion and comes in close contact with an affected area of the body, a protective film portion which is attached to a lower surface of the adhesive portion to cover the gauze portion and is separated from the adhesive portion during attachment of the sheet portion to the body, and a grip portion which protrudes from one side of the sheet portion in order to be gripped by the user and is rotatably connected to the one side of the sheet portion with respect to a fold line.

Also, the dressing band may further include an information input portion which has a predetermined area so that information is able to be written thereon and which is attached to an upper surface of the sheet portion, and the information input portion may be eccentrically positioned at one side edge of the sheet portion.

Also, the fold line may have a length along an edge of the sheet portion, and the grip portion may protrude in a direction opposite to the information input portion with respect to a width direction of the fold line.

Also, the sheet portion may have the shape of a quadrangular pad which has four corner portions formed along side surfaces, and the grip portion may be eccentrically positioned in a direction in which the corner portions are connected.

Also, one or more cut portions which are penetrated in a vertical direction may be formed in the edge of the sheet portion, the cut portion may have a length along both sides, and one side of the cut portion in a longitudinal direction thereof may be open toward the edge of the sheet portion.

According to the present invention, a sheet portion can be easily peeled off from the user's body, a risk of infection can be reduced because contaminants do not get on an adhesive portion upon separation of a protective film portion, and hygienic use is possible because the adhesive portion and the contaminants do not get on the user's hand in a process of peeling off the sheet portion.

Also, according to the present invention, safety in use can be secured because there is no concern about causing additional wounds and infection in the process of peeling off the sheet portion, various pieces of information (date, time, condition, etc.) can be easily checked because such information can be written on an information input portion, and additional wounds (skin problems or the like) can be prevented because a replacement time, a peeling time, or the like of the sheet portion can be checked by visual inspection.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is an exploded perspective view for showing a dressing band according to the present invention;

FIG. 2 is a bottom view for showing the dressing band according to the present invention;

FIG. 3 is a lateral view for showing a process of peeling off a protective film portion of the dressing band according to the present invention;

FIG. 4 is a use state view for showing a state in which a sheet portion of the dressing band according to the present invention is attached to the human body;

FIG. 5 is an exploded perspective view for showing a state in which an information input portion is applied to the dressing band according to the present invention;

FIG. 6 is a bottom view for showing the state in which the information input portion is applied to the dressing band according to the present invention;

FIG. 7 is a use state view for showing a state in which the information input portion is applied to the dressing band according to the present invention and the sheet portion is attached to the human body;

FIG. 8 is a lateral view for showing a process in which the information input portion is applied to the dressing band according to the present invention and the protective film portion is peeled off;

FIG. 9 is a lateral view for showing a process in which the information input portion is applied to the dressing band according to the present invention and the sheet portion is separated from the human body;

FIG. 10 is an exploded perspective view for showing a state in which a cut portion is applied to the sheet portion of the dressing band according to the present invention;

FIG. 11 is a bottom view for showing the state in which the cut portion is applied to the sheet portion of the dressing band according to the present invention;

FIG. 12 is an exploded perspective view for showing a state in which a grip portion of the dressing band according to the present invention is disposed as a pair of grip portions at both sides of the sheet portion;

FIG. 13 is a lateral view for showing a state in which spacing protrusions are formed on the grip portion of the dressing band according to the present invention;

FIG. 14 is a lateral view for showing a state in which a cover portion is attached to an upper portion of the information input portion in the dressing band according to the present invention;

FIG. 15 is a perspective view for showing a state in which an auxiliary adhesive portion is applied on the grip portion of the dressing band according to the present invention; and

FIG. 16 is a perspective view for showing a state in which a fixing member is coupled to the sheet portion of the dressing band according to the present invention.

### DETAILED DESCRIPTION OF EXEMPLARY EMBODIMENTS

Hereinafter, exemplary embodiments according to the present invention will be described in detail with reference to the accompanying drawings.

Advantages and features of the present invention and a method of achieving the same should become clear with embodiments described in detail below with reference to the accompanying drawings.

However, the present invention is not limited by embodiments disclosed below and is realized in various different forms. The present embodiments make the disclosure of the present invention complete and are provided to completely inform one of ordinary skill in the art to which the present invention pertains of the scope of the disclosure. The present invention is defined only by the scope of the claims.

In addition, in describing the present invention, when a known related art or the like is considered as having the possibility of obscuring the gist of the present invention, detailed description thereof will be omitted.

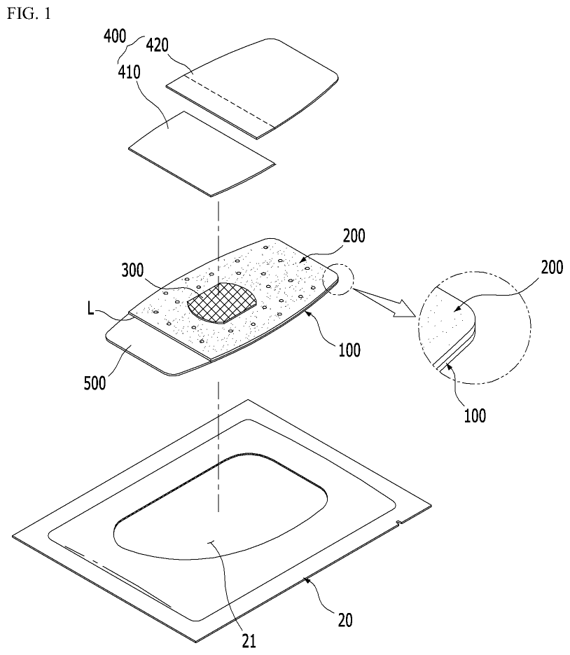

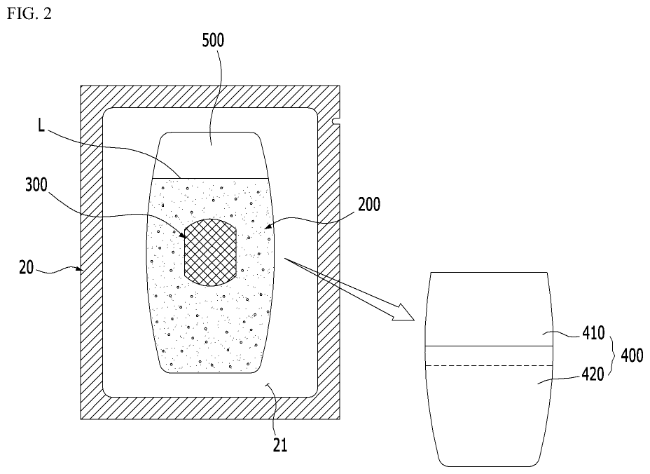

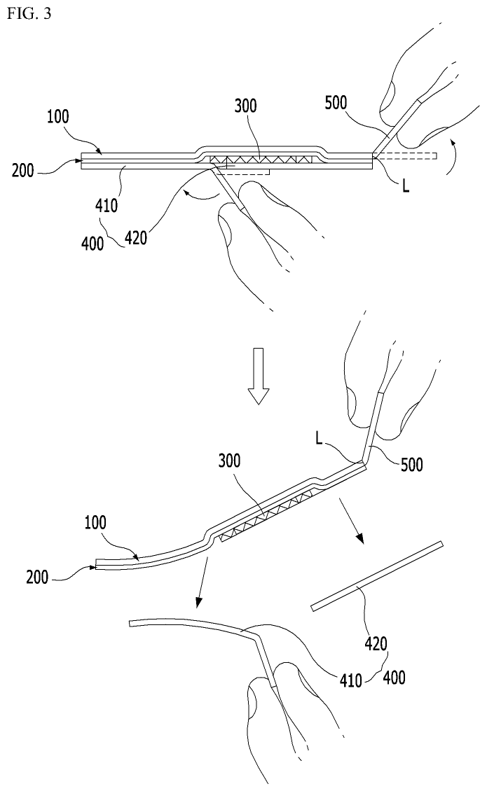

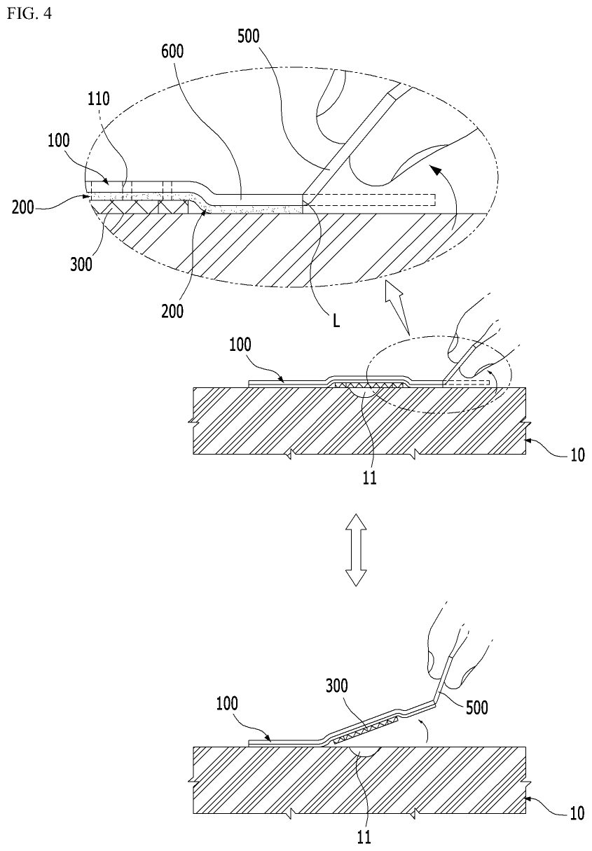

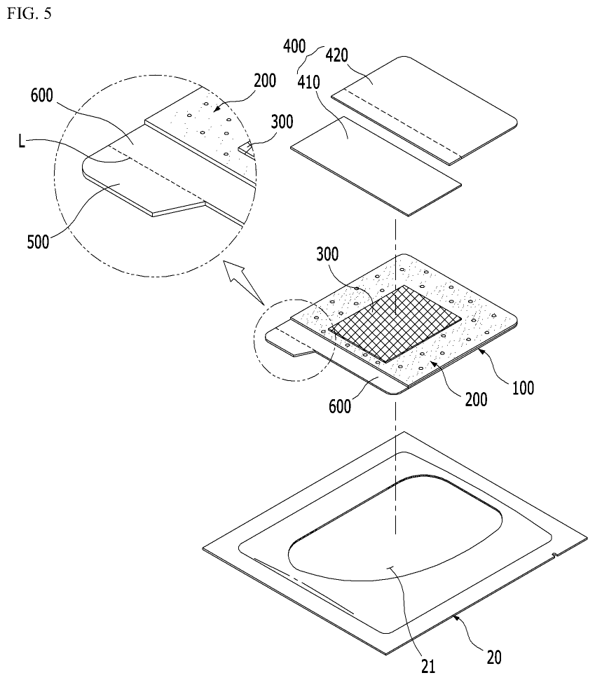

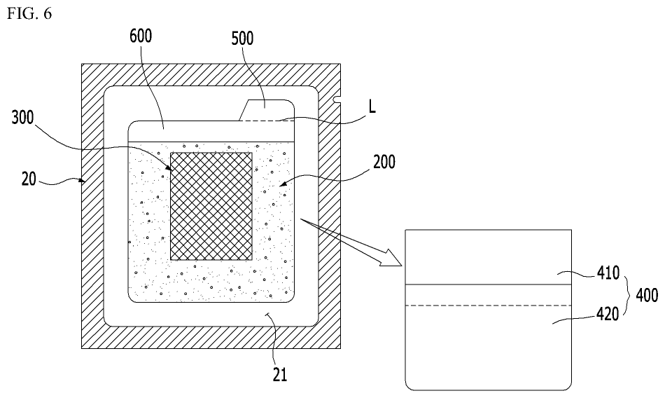

FIG. 1 is an exploded perspective view for showing a dressing band according to the present invention, FIG. 2 is a bottom view for showing the dressing band according to the present invention, and FIG. 3 is a lateral view for showing a process of peeling off a protective film portion of the dressing band according to the present invention. Also, FIG. 4 is a use state view for showing a state in which a sheet portion of the dressing band according to the present invention is attached to the human body, FIG. 5 is an exploded perspective view for showing a state in which an information input portion is applied to the dressing band according to the present invention, and FIG. 6 is a bottom view for showing the state in which the information input portion is applied to the dressing band according to the present invention.

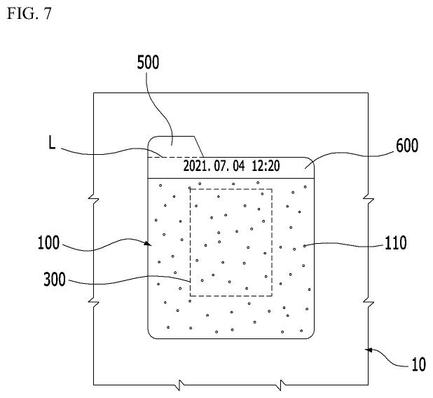

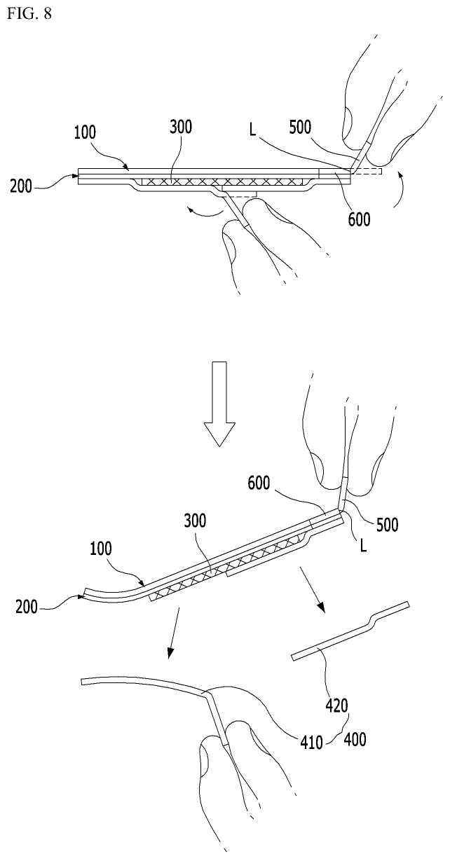

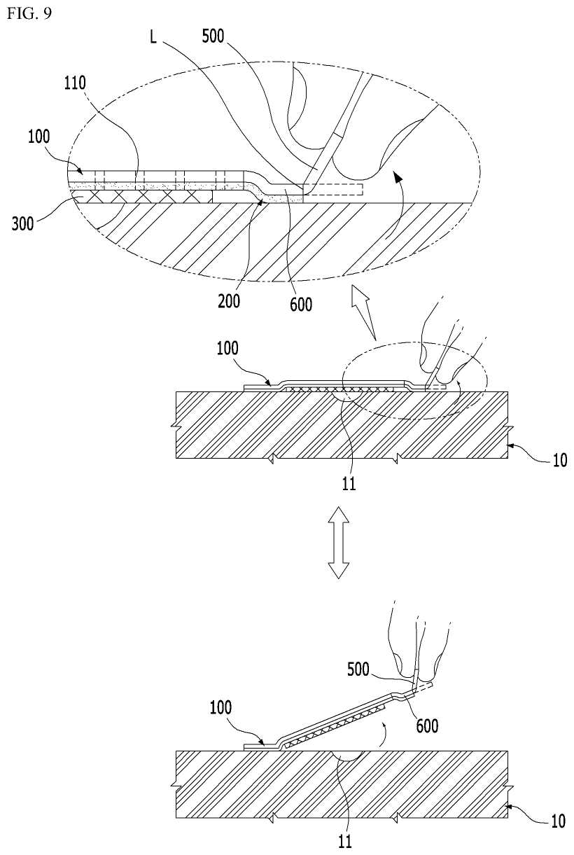

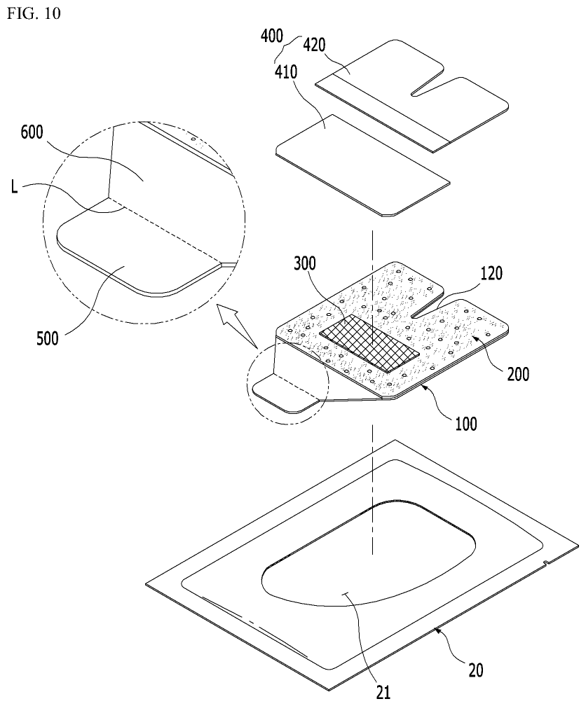

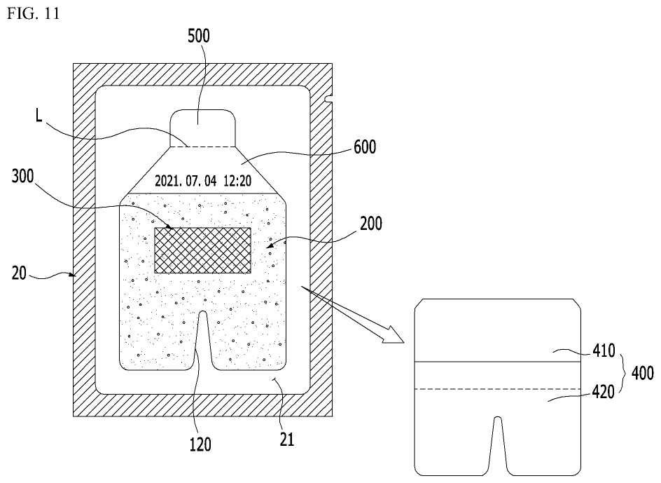

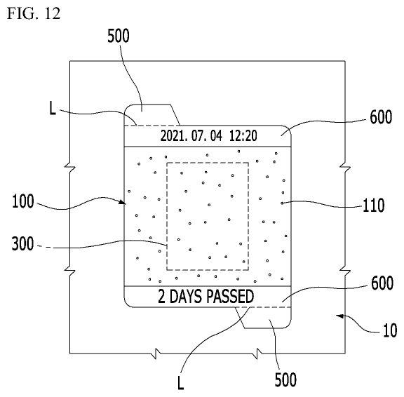

FIG. 7 is a use state view for showing a state in which the information input portion is applied to the dressing band according to the present invention and the sheet portion is attached to the human body, FIG. 8 is a lateral view for showing a process in which the information input portion is applied to the dressing band according to the present invention and the protective film portion is peeled off, and FIG. 9 is a lateral view for showing a process in which the information input portion is applied to the dressing band according to the present invention and the sheet portion is separated from the human body. Also, FIG. 10 is an exploded perspective view for showing a state in which a cut portion is applied to the sheet portion of the dressing band according to the present invention, FIG. 11 is a bottom view for showing the state in which the cut portion is applied to the sheet portion of the dressing band according to the present invention, and FIG. 12 is an exploded perspective view for showing a state in which a grip portion of the dressing band according to the present invention is disposed as a pair of grip portions at both sides of the sheet portion.

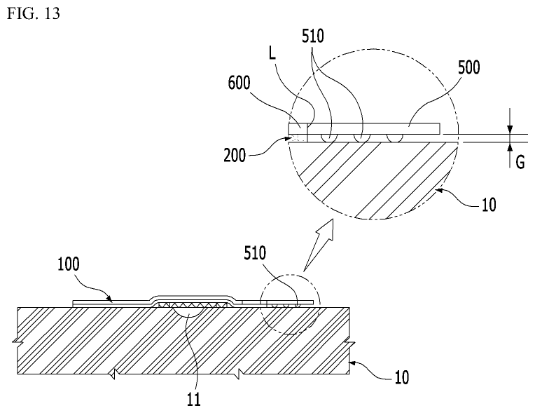

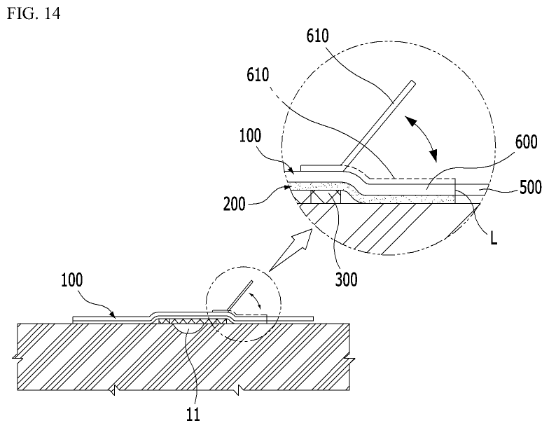

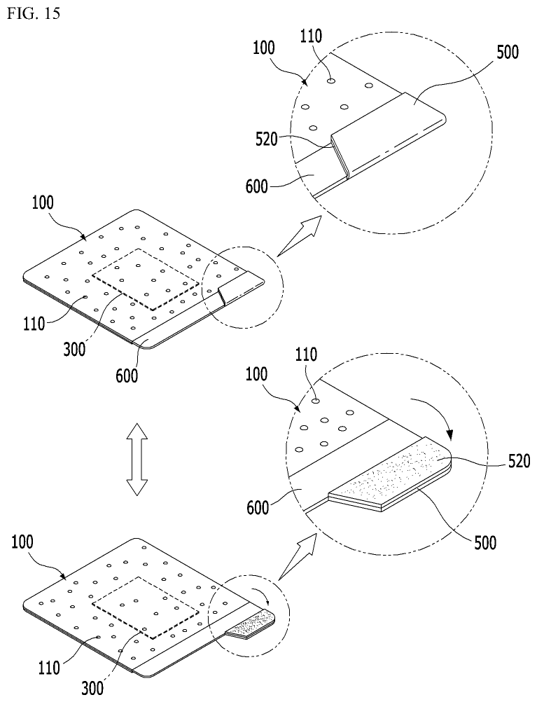

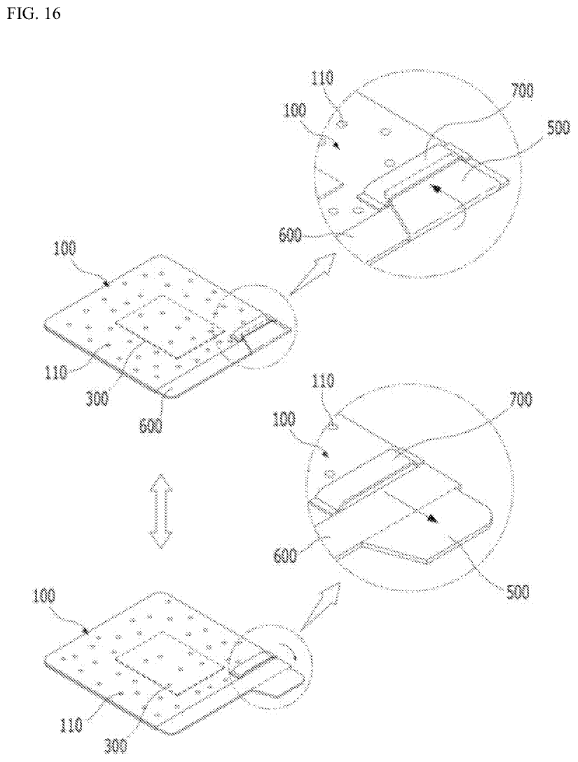

FIG. 13 is a lateral view for showing a state in which spacing protrusions are formed on the grip portion of the dressing band according to the present invention, FIG. 14 is a lateral view for showing a state in which a cover portion is attached to an upper portion of the information input portion in the dressing band according to the present invention, and FIG. 15 is a perspective view for showing a state in which an auxiliary adhesive portion is applied on the grip portion of the dressing band according to the present invention. Also, FIG. 16 is a perspective view for showing a state in which a fixing member is coupled to the sheet portion of the dressing band according to the present invention.

Referring to FIGS. 1 to 16, the dressing band according to the present invention includes a sheet portion 100, an adhesive portion 200, a gauze portion 300, a protective film portion 400, one or more grip portions 500, and one or more information input portions 600.

The sheet portion 100 is a configuration to come in close contact with a user's body 10 (skin or the like) due to the adhesive portion 200 which will be described below. The sheet portion 100 comes in close contact with the body 10 and covers an affected area 11 from the outside and may be wrapped in a state in which it is sealed in an accommodation space 21 of a separate wrapping portion 20 (vinyl or the like). Here, the sheet portion 100 may have a lower surface that comes in close contact with the body 10 and may be made to have a certain area using an elastic material so that the sheet portion 100 is deformable to a shape that corresponds to the body 10. Here, materials such as a nonwoven fabric, polyurethane (PU), and silicone may be selectively used for the sheet portion 100.

Also, the sheet portion 100 may be made in the shape of a quadrangular pad that has four corner portions formed along side surfaces or may be made in the shape of a circular pad which has a curvature formed at a lateral end. A plurality of air holes 110 may be formed to vertically pass through the sheet portion 100 to allow air to flow in a vertical direction.

Also, at an edge of the sheet portion 100, as illustrated in FIG. 11, one or more cut portions 120 may be formed to be penetrated in a vertical direction, have a length along both sides, and have one longitudinal side open toward the edge of the sheet portion 100. An auxiliary sheet portion 130 may be disposed at each of both sides with respect to a width direction of the cut portion 120. The auxiliary sheet portion 130 may have one end connected to the edge of the sheet portion 100 and the other end extending in a direction opposite to the sheet portion 100.

That is, since the auxiliary sheet portions 130 are positioned in a state in which they are divided from each other by the cut portion 120, attachment positions of the auxiliary sheet portions 130 may be separately adjusted, and since the shapes, lengths, or the like of the auxiliary sheet portions 130 may be adjusted to be different from each other, convenience in use can be improved.

The adhesive portion 200 is for attaching the sheet portion 100 to the user's body 10. The adhesive portion 200 may be applied with a certain thickness on a lower surface of the sheet portion 100 and attached to the body 10, and a medical adhesive or the like that is harmless to the human body may be selectively used for the adhesive portion 200. Here, the adhesive portion 200 may be applied on the entire area of the sheet portion 100, but a form in which the adhesive portion 200 is applied may be changed in various ways as necessary.

The gauze portion 300 is a portion that comes in close contact with the affected area 11 of the body 10 and may be attached to the lower surface of the sheet portion 100. Fabric, cotton, or the like that is harmless to the human body may be used for the gauze portion 300, and the gauze portion 300 may contain a drug. The shape, size, material, or the like of the gauze portion 300 may be changed in various ways as necessary.

The protective film portion 400 is attached to a lower surface of the adhesive portion 200 and covers the gauze portion 300 from below and is separated from the adhesive portion 200 during attachment of the sheet portion 100 to the body 10. Here, the protective film portion 400 may be made using a material such as transparent vinyl, but the color, material, or the like of the protective film portion 400 may be changed in various ways as necessary.

Also, the protective film portion 400 may include a first protective film 410 which has a certain area and is attached to one side of the adhesive portion 200 and a second protective film 420 which has a certain area and is attached to the adhesive portion 200 at a position that corresponds to the first protective film 410. Here, one end of the first protective film 410 and one end of the second protective film 420 that face each other may be positioned in a state in which they overlap each other in the vertical direction in order to be gripped by a user's hand.

The grip portion 500 is a configuration to be gripped by a user's hand and protrudes from one side of the sheet portion 100 in order to be gripped by the user, and is rotatably connected to the one side of the sheet portion 100 with respect to a fold line L. Here, the grip portion 500 may protrude with a width narrower than a width of the sheet portion 100. Here, a marking surface may be formed on an upper surface of the grip portion 500 to allow various pieces of information to be written thereon with a pen or the like, a material such as paper may be attached to the marking surface, and a material identical to a material of the information input portion 600 which will be described below may be attached to the marking surface.

Also, the grip portion 500 may be positioned to correspond to both sides of the sheet portion 100 as illustrated in FIG. 12. For example, in the case in which the sheet portion 100 is made in the shape of a quadrangular pad, each of a pair of grip portions 500 may be eccentrically positioned at one side of a corner portion of the sheet portion 100. That is, since the user may use the grip portion 500 positioned to be easily gripped by a hand, convenience in use can be secured.

The fold line L may be formed along an edge of the sheet portion 100, one side of the grip portion 500 and one side of the sheet portion 100 may be connected to each other with respect to the fold line L, and the fold line L may be formed with a vertical thickness narrower than a vertical thickness of the sheet portion 100 to facilitate rotation of the grip portion 500.

In the case in which the sheet portion 100 is made in the shape of a quadrangular pad, the grip portion 500 may be eccentrically positioned at one side of a corner portion of the sheet portion 100 as illustrated in FIGS. 5 to 16. For example, in a case in which the sheet portion 100 attached to the body 10 is separated, the user may rotate the grip portion 500 in a direction opposite to the body 10 and then grip the grip portion 500 by hand, pull the grip portion 500 in the direction opposite to the body 10, and sequentially separate the sheet portion 100.

That is, since the sheet portion 100 is sequentially separated from a vertex portion where corner portions of the sheet portion 100 meet (a portion where an area of contact is narrow), the sheet portion 100 may be easily separated without much effort, and since a state in which the grip portion 500 is separated from the body 10 is maintained, the user can easily grip the grip portion 500.

In the dressing band according to one embodiment of the present invention, one or more spacing protrusions 510 may protrude from a lower surface of the grip portion 500 as illustrated in FIG. 13. In a case in which the sheet portion 100 is attached to the body 10, protruding ends of the spacing protrusions 510 may support the body 10, and the protruding ends of the spacing protrusions 510 may form a curvature (a semi-spherical shape or the like) that protrudes to prevent pain upon contact with the body 10.

Also, a gap G may be formed between the body 10 and the grip portion 500 by as much as a length at which the spacing protrusions 510 protrude, and the user may put his or her finger in the gap G and then rotate the grip portion 500 in the direction opposite to the body 10. That is, since the spacing protrusions 510 come in contact with the body 10 through a narrow area, an area of contact with the body 10 may be reduced, and since the gap G is formed, air permeability can be secured. Also, since the gap G in which the user may put his or her finger is secured, the grip portion 500 can be easily gripped.

The information input portion 600 is a configuration to write thereon the date and/or time at which the sheet portion 100 is attached to the body 10, a condition of the affected area 11, and the like. The information input portion 600 may have a certain area and be attached to an upper surface of the sheet portion 100, and a material such as paper may be used for the information input portion 600 to allow various pieces of information to be written thereon with a pen or the like.

Also, the information input portion 600 may be eccentrically positioned at one side edge of the sheet portion 100, the fold line L may have a length along the edge of the sheet portion 100, and the grip portion 500 may protrude in a direction opposite to the information input portion 600 with respect to a width direction of the fold line L.

Also, in the dressing band according to one embodiment of the present invention, as illustrated in FIG. 14, a cover portion 610 may be further disposed on an upper portion of the information input portion 600 in a state in which the cover portion 610 is rotatable and openable/closeable. The cover portion 610 may have one side rotatably attached to the upper surface of the sheet portion 100 and the other side rotatably provided. Here, the cover portion 610 may be made using a transparent material (vinyl or the like) so that various pieces of information written on the upper surface of the information input portion 600 are visible from the top.

For example, in a case in which the cover portion 610 is rotated in a direction toward the information input portion 600, since the cover portion 610 covers the upper surface of the information input portion 600, various pieces of information (date, time, condition, etc.) written on the upper surface of the information input portion 600 may be not exposed to the outside, and the cover portion 610 may be opened only when writing information on the upper surface of the information input portion 600 with a pen or the like. That is, since the information input portion 600 is covered by the cover portion 610, a raw material (ink or the like) used to write on the information input portion 600 can be prevented from staining clothes or the like, and the raw material can be prevented from spreading due to moisture or the like.

Also, in the dressing band according to one embodiment of the present invention, as illustrated in FIG. 15, an auxiliary adhesive portion 520 for fixing the grip portion 500 may be further applied on the upper surface of the grip portion 500. The auxiliary adhesive portion 520 is for fixing the grip portion 500 in a folded state on the upper surface of the information input portion 600, and in a case in which the grip portion 500 is brought into close contact with the upper surface of the information input portion 600 in a direction toward the sheet portion 100, the auxiliary adhesive portion 520 is attached to the upper surface of the information input portion 600.

Here, the auxiliary adhesive portion 520 may be applied with a certain thickness on the lower surface of the grip portion 500 and attached to the upper surface of the information input portion 600, and a medical adhesive or the like that is harmless to the human body may be selectively used for the auxiliary adhesive portion 520. Here, the auxiliary adhesive portion 520 may be applied on the entire area of the grip portion 500, but the form in which the auxiliary adhesive portion 520 is applied may be changed in various ways as necessary.

For example, in a case in which the sheet portion 100 is wrapped for sale, since the grip portion 500 in a folded state may be brought into close contact with the upper surface of the information input portion 600 and the grip portion 500 remains attached to the upper surface of the information input portion 600 due to an adhesive force of the auxiliary adhesive portion 520, the volume of the sheet portion 100 may be reduced, and since the grip portion 500 does not longitudinally protrude from an outer boundary of the sheet portion 100, the sheet portion 100 can be easily stored in the accommodation space 21 of the wrapping portion 20.

On the other hand, in a case in which the sheet portion 100 is separated from the wrapping portion 20 and attached to the affected area 11 of the body 10, the user may separate the grip portion 500 from the upper surface of the information input portion 600 and then unfold the grip portion 500 toward the outer boundary of the sheet portion 100. That is, the grip portion 500 may protrude only when attaching the dressing band to the affected area 11.

Also, in the dressing band according to one embodiment of the present invention, as illustrated in FIG. 16, a fixing member 700 for fixing the grip portion 500 may be further disposed on the upper surface of the sheet portion 100. Both ends of the fixing member 700 may be coupled to the upper surface of the sheet portion 100, an insertion groove may be formed between the fixing member 700 and the sheet portion 100, and the insertion groove may be horizontally open in a direction toward the grip portion 500.

For example, in the case in which the sheet portion 100 is wrapped for sale, since one end of the grip portion 500 may be inserted into the insertion groove after the grip portion 500 is rotated in a direction toward the upper surface of the sheet portion 100, and the grip portion 500 remains folded, the volume of the sheet portion 100 may be reduced, and since the grip portion 500 does not longitudinally protrude from the outer boundary of the sheet portion 100, the sheet portion 100 can be easily stored in the accommodation space 21 of the wrapping portion 20.

On the other hand, in a case in which the sheet portion 100 is separated from the wrapping portion 20 and attached to the affected area 11 of the body 10, the user may separate the one end of the grip portion 500 from the insertion groove and then unfold the grip portion 500 toward the outer boundary of the sheet portion 100. That is, the grip portion 500 may protrude only when attaching the dressing band to the affected area 11.

Consequently, according to the present invention, it is possible to easily peel off the sheet portion 100 from the user's body 10, contaminants do not get on the adhesive portion 200 upon separation of the protective film portion 400, thus reducing a risk of infection, and the adhesive portion 200 and the contaminants do not get on the user's hand in a process of peeling off the sheet portion 100, thus allowing hygienic use.

Also, according to the present invention, there is no concern about causing additional wounds and infection in the process of peeling off the sheet portion 100, thus securing safety in use, various pieces of information (date, time, condition, etc.) may be written on the information input portion 600, thus allowing such information to be easily checked, and the sheet portion 100 can be prevented from being attached to the body for a long period of time, thus preventing additional wounds (skin problems or the like).

Specific embodiments relating to the dressing band according to the present invention have been described above, but it should be apparent that various modifications are possible within the scope not departing from the scope of the present invention.

Therefore, the scope of the present invention should not be defined as being limited to the embodiments described herein and should be defined by the attached claims and their equivalents.

That is, the embodiments described herein should be understood as being illustrative, instead of limiting, in all aspects. The scope of the present invention is shown by the attached claims rather than by the detailed description above, and all changes or modifications derived from the meaning and scope of the claims and their equivalents should be construed as belonging to the scope of the present invention.

What is claimed is:

1. A dressing band comprising:
a sheet portion whose lower surface comes in close contact with a user's body;
an adhesive portion which is applied on the lower surface of the sheet portion and attached to the body;
a gauze portion which is attached to the lower surface of the sheet portion and comes in close contact with an affected area of the body;
a protective film portion which is attached to a lower surface of the adhesive portion to cover the gauze portion and is separated from the adhesive portion during attachment of the sheet portion to the body; and
a grip portion which protrudes from one side of the sheet portion in order to be gripped by the user and is rotatably connected to the one side of the sheet portion with respect to a fold line,
wherein the dressing band further includes an information input portion which has a predetermined area so that information is able to be written thereon and which is attached to an upper surface of the sheet portion, and the information input portion is eccentrically positioned at one side edge of the sheet portion,
the fold line has a length along an edge of the sheet portion, and the grip portion protrudes in a direction opposite to the information input portion with respect to a width direction of the fold line,
the grip portion is positioned to correspond to both sides of the sheet portion,
one or more spacing protrusions protrude from a lower surface of the grip portion, in a case in which the sheet portion is attached to the body, protruding ends of the spacing protrusions support the body, and the protruding ends of the spacing protrusions form a curvature that protrudes, and
an auxiliary adhesive portion is further applied on an upper surface of the grip portion, and in a case in which the grip portion is brought into close contact with an upper surface of the information input portion, the auxiliary adhesive portion is attached to the upper surface of the information input portion.

2. The dressing band of claim 1, wherein:
the sheet portion has the shape of a quadrangular pad which has four corner portions formed along side surfaces; and
the grip portion is eccentrically positioned in a direction in which the corner portions are connected.

3. The dressing band of claim 1, wherein:
one or more cut portions which are penetrated in a vertical direction are formed in an edge of the sheet portion; and
the cut portion has a length along both sides, and one side of the cut portion in a longitudinal direction thereof is open toward the edge of the sheet portion.
